(19) United States
(12) Patent Application Publication
(10) Pub. No.: US 2020/0286580 A1
(43) Pub. Date: Sep. 10, 2020

(54) SYSTEM AND METHOD FOR ENGINEERING, TESTING AND MODELLING A BIOLOGICAL CIRCUIT

(71) Applicant: INSTITUTE OF SCIENCE AND TECHNOLOGY AUSTRIA (IST AUSTRIA), Klostemeuburg (AT)

(72) Inventors: Remy CHAIT, Klosterneuburg (AT); Ruess JAKOB, Klosterneuburg (AT); Lang MORITZ, Klosterneuburg (AT); Guet CALIN, Klosterneuburg (AT); Gasper TKACIK, Klosterneuburg (AT)

(21) Appl. No.: 16/759,484

(22) PCT Filed: Oct. 29, 2018
(86) PCT No.: PCT/EP2018/079597

§ 371 Date: Apr. 27, 2020

(30) Foreign Application Priority Data
Oct. 30, 2017 (GB) 1717821.1

(51) Int. Cl. G16B 5/20; C12Q 3/00

(52) U.S. Cl. CPC G16B 5/20; C12Q 3/00

(57) ABSTRACT

The invention relates to systems and methods for engineering, testing or modelling a biological circuit. The biological circuit is split into two parts, wherein one part is implemented in one or more living cells and the other part is simulated on a computer and the two parts interface via a closed loop in real time. The system comprises (i) means for culturing one or more living cells; (ii) one or more living cells in which a cellular part of the biological circuit is implemented; (iii) means for controlling the state or environment of the cell(s); (iv) means for taking measurements of one or more cell state or environmental parameters from the cell(s); and (v) a computer. The computers simulates the remaining part of the biological circuit in real time; and the two parts interface via a closed loop in which the output from the simulation provides input into the cellular part of the biological circuit via the controlling means; and cell state or environmental parameter measurements taken from the cell(s) provide input into the simulated part of the circuit.

### FIELD OF THE INVENTION

The invention relates to systems and methods for engineering, testing or modelling a biological circuit.

### BACKGROUND OF THE INVENTION

Biomolecular networks composed of interacting genes, sensors, metabolites, and similar components govern the behaviour and products of biological cells as well as the interactions between individual cells. However, even simple biomolecular networks can be extremely challenging to understand, predict and construct, rendering the industrial or scientific design and realization of synthetic signalling or metabolic pathways slow and inefficient. The reasons for this inefficiency include poorly characterized or unknown interactions of genes with other genes, metabolites, with the cellular milieu, and with the cell's external environment. Testing which of these possibilities apply to a given synthetic network under construction is tedious and expensive. Consequently, a significant proportion of the time and cost of developing synthetic network is spent in “debugging”, that is, in the successive identification of unanticipated interactions that render a given network design non-functional when implemented in situ. Theoretical models can assist in this process by helping to interpret experimental data and by suggesting new experiments and perturbations to perform. Based on insights gained from mathematical modelling after each round of experiments, new experiments can be performed which, again, serve to redefine the mathematical model. However, this traditional development cycle runs profoundly slowly due to the long time scales required to implement and validate changes in all but the most trivial biomolecular networks in situ.

### SUMMARY OF THE INVENTION

The inventors have developed systems and methods for engineering, testing or modelling a biological circuit that approach these problems in a new way. The invention is centred on the novel insight that not all components of a synthetic biomolecular network have to be implemented at once and/or in the same cell at intermediate stages of the implementation of the network. Instead, only sub-networks of different size and complexity (alternatively referred to in the following as “units”, “parts”, “sub-circuits” or “modules”), which might range from single genes to nearly the complete network, may be implemented in situ in each implementation stage, while the rest of the network is simulated in silico based on the current specification of the network. Interactions between the parts of a network implemented in situ (the cellular part) and the rest of the network simulated in silico (the simulated part) are realized by measuring the current state of the cellular part in the form of cellular, metabolic or environmental readouts (fluorescence proteins, cell segmentation, mass spectroscopy, and similar), and feeding back the current state of the computer simulation of the rest of the network using various experimentally definable cellular, metabolic or environmental inputs (chemical environment, optogenetic inputs, and similar). By thus “virtualizing” the interactions of the cellular part with the rest of the network while still allowing for all interactions (anticipated or not) of the cellular part with the rest of the cellular and extracellular environment, such a framework can dramatically speed up implementation and characterization of biomolecular networks in situ.

Specifically, advantages provided by the present invention include (i) unanticipated or poorly-characterized interactions of genes with other genes, metabolites, with the cellular milieu, the cell's external environment, and similar can be efficiently identified since these interactions have to occur in or involving only the units already implemented in situ; (ii) correct functioning of individual units in the context of the whole (synthetic) biomolecular network can be tested and its dynamic functionality assessed even before all parts of the network are implemented; this enables better predictions of how a unit will perform if it is implemented into a larger network in situ and faster and more economical laboratory development cycles; (iii) broad sets of novel design features, such as interactions and sensors, can be quickly simulated and virtually integrated into an existing or already partly implemented core network, allowing to evaluate their performance directly in situ for targeted development; (iv) different units can be implemented in different cells or by different persons in parallel, potentially in different laboratories and on different continents, while still allowing realistic testing of the interactions between these units; (v) different units can be easily composed into larger units, and a larger unit can be split up into several smaller units, allowing developers to dynamically change the size of the subnetwork which is tested during a given implementation stage; and (vi) different scenarios can be tested under actual biological conditions including extrinsic- and intrinsic noise, cell and population growth and cellular variability. Such capabilities can iteratively guide the construction of gene networks and external conditions required for optimal operation, and allow exploration of the behaviour of bio-digital hybrid networks with setup times in the minutes or hours to days rather than weeks to months or years typically required of testing purely biological systems.

In a first aspect the invention provides a system for engineering, testing or modelling a biological circuit, the system comprising
(i) means for culturing one or more living cells;
(ii) one or more living cells in which a cellular part of the biological circuit is implemented;
(iii) means for controlling the state or environment of the cell(s);
(iv) means for taking measurements of one or more cell state or environmental parameters from the cell(s); and
(v) a computer;
   characterised in that the computers simulates the remaining part of the biological circuit in real time; and the two parts interface via a closed loop in which the output from the simulation provides input into the cellular part of the biological circuit via the controlling means; and cell state or environmental parameter measurements taken from the cell(s) provide input into the simulated part of the circuit.

In a further aspect the invention also provides the use of the system for engineering, testing or modelling a biological circuit.

In a still further aspect the invention provides a method of engineering, testing or modelling a biological circuit, the method comprising
(i) culturing one of more living cells, wherein a cellular part of the biological circuit is implemented in the cell(s);
(ii) taking measurements of one or more cell state or environmental parameters from the cell(s);
(iii) simulating the remaining part of the biological circuit in real time on a computer; and
(iv) interfacing the cellular and simulated parts of the circuit via a closed loop in which the output from the simulation provides input into the cellular part of the biological circuit via means for controlling the environment or state of the cell(s); and cell state or environmental parameter measurements taken from the cell(s) provide input into the simulated part of the biological circuit.

The invention will now be described in more detail, by way of example and not limitation, and by reference to the accompanying drawings. Many equivalent modifications and variations will be apparent, to those skilled in the art when given this disclosure. Accordingly, the exemplary embodiments of the invention set forth are considered to be illustrative and not limiting. Various changes to the described embodiments may be made without departing from the scope of the invention. All documents cited herein, whether supra or infra, are expressly incorporated by reference in their entirety.

The present invention includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “a gene” includes two or more such genes.

Section headings are used herein for convenience only and are not to be construed as limiting in any way.

### BRIEF DESCRIPTION OF THE DRAWINGS

FIG. 1. An experimental platform for independently-programmable optogenetic control of gene expression in individual bacteria.

a. Online measurement and control (cell out-, in-arrows) coupled through an experimental platform (f) of many single bacteria at once enables probes of single cell heterogeneity, effects of environmental variability in individual cells, and group behaviors of interacting individuals. b. Experimental platform overview: Individual bacteria are cultured for days in defined chemical environments within a microfluidic “mother machine” device. Fluorescent reporter expression, cell-shape, and growth rate data are automatically captured from fluorescent microscope images and provided to each cell's individually-specified software controller. The controllers output stimuli to up- or down-regulate a light-responsive gene for each cell. The individual stimuli are collected, spatially arranged and transmitted to the recipient cells using a custom modified microscope-coupled LCD projector. This process is repeated every 6 minutes. c. Cerulean CFP expressed via an optimized CcaSR optogenetic regulation system (Schmidl, S. R., Sheth, R. U., Wu, A. & Tabor, J. J. Refactoring and Optimization of Light-Switchable Escherichia coli Two-Component Systems. ACS Synth. Biol. 3, 820-831 (2014)). CcaS-phycocyanobilin autophosphorylates under green light (535 nm), then phosphorylates CcaR, which binds and activates expression from the PcpcG2-172 promoter. Exposure to red light (670 nm) dephosphorylates CcaS, eventually halting expression.

FIG. 2. Parallelized model predictive control of individual cells.

Single cell controllers iteratively use measured fluorescence trajectories (examples for three individual cells, top) and a Kalman filter to infer cells' transcriptional responsiveness, E(t), based on past activating and deactivating light sequences (green (light grey), red (dark grey) series), and suggest the next stimuli from light sequences that minimize the error between expected future fluorescence levels and a target profile (red line) within a specified planning horizon. To calculate expected fluorescence levels and infer responsiveness, the controllers make use of a simple stochastic model of gene expression (gray box, bottom right) consisting of three state variables that represent light-activation H(t), cell responsiveness E(t), and fluorescence F(t). The same control scheme could use different, potentially more complex, models of gene expression, by simply swapping the content of the “Model” module.

FIG. 3. Specifying gene expression distributions in small bacterial populations using iCL control.

a. CFP concentration trajectories (grey lines) for individual Escherichia coli cells tracking target trajectories (red lines) under model-based control in either open loop (left panel, n=55 cells) or individual closed loop mode (right panel, n=49 cells). Data are pooled from two replicate experiments. Deviations of average population trajectories (blue lines) from targets represent errors in control of the mean, while breadth of the expression distributions (shaded regions, +/−1 s.d.) indicate errors in control of individual cells. Individual closed loop control reduces mean error and cell-to-cell variation, and removes extended excursions in responsiveness seen in open loop (e.g., asterisk-419 labeled CFP trajectory).

b. Mean CFP trajectories+/−one standard deviation (dark lines and dark grey, light grey-shaded regions, mean trajectories smoothed with 1 hour moving average) for cells controlled towards fluorescence targets (red lines) of either 10 a.u. (n=18 cells), or 20 a.u. (n=14 cells). Control for all cells switches from open loop (orange-labelled time interval) to individual closed loop (blue interval) mode at 780 min. Individual light stimulation sequences (green (light grey): activation, red (dark grey): repression) for cells targeted to 20 a.u. (upper sequences) and 10 a.u. (lower sequences) are displayed below the plot. Probability distributions of CFP levels in open and individual closed loop regimes (OL, iCL shaded intervals, respectively) are shown to the right. With individual closed loop control (right plot), cells are resolved into target group distributions (dark grey, light grey distributions, smoothed: dashed lines) by reducing large errors between target group means (triangles) and expression targets (red lines), and narrowing individual errors (error bars: +/−1 s.d.). c. Average power spectral density (orange) of open loop controlled fluorescence trajectories (OL shaded interval) in (b). Fluctuations at frequencies below 0.02 min⁻¹ are reduced in the iCL shaded interval under individual closed loop control (blue). Average estimates of power spectra are for 14 mean-subtracted trajectories with constant fluorescence target of 20 a.u. (spectra are similar for 18 cells targeting 10 a.u.). Dashed lines denote standard error of the mean. d. Single-cell temporal patterning of CFP expression in 24 Escherichia coli cells. Individual closed loop control targets are raster of a binarized image (inset) with fluorescence levels 0 and 15 a.u. The control interval is 24 hours, following 10 hour pre-control adaptation interval (not displayed). Cells shown are automatically selected by longest-validity (Methods) from 8 simultaneous replicates.

FIG. 4. iCL control maintains low mean and individual errors in a population perturbed with antibiotic.

a. Growth rates (individual: light lines; mean: dark lines) of cells under open loop (OL) control (orange, n=40 cells), and individual closed loop (iCL) control (light blue, n=36 cells). Growth slows after 0.6 ug/ml doxycycline is added to the culture media at 600 min (vertical line). b. Individual and mean CFP fluorescence trajectories (grey, blue lines respectively; green shaded regions (between thin dark lines)=mean+/−1 s.d.) of cells tracking a constant target (15 a.u.) under OL control (upper panel) or iCL control (lower panel). Data are normalized, per controller, to mean fluorescence levels (red dashed lines) within a 5-hour interval immediately prior to doxycycline exposure (−Dox, shaded region), for comparison to a second interval (+Dox, shaded region) between 10 and 15 hours after doxycycline addition. c. Perturbation of pre-antibiotic (−Dox shaded region in (b)) mean-normalized CFP fluorescence (red dashed line) by doxycycline (+Dox shaded region in (b)), shown by box plot distributions for open loop (OL, orange) or individual closed loop (iCL, blue) controlled cells.

FIG. 5. Digitally specified communication between single-cell hybrid oscillators.

a. Simplified scheme for a biological oscillator driven by a delayed negative feedback loop (Novak & Tyson, “Design principles of biochemical oscillator” Nat. Rev. Mol. Cell Biol. Vol, 9, pp. 981-991, 2008) on expression of an enzyme (ENZ) by an enzyme-produced signal (S). b. Analogous architecture of a hybrid oscillator with virtualized signal (S). A CFP expression reporter and light-responsive promoter interface the e biological side of the circuit to a digital component that implements a discrete ti 457 me CFP-dependent signal accumulation (production rate r, removal rate d), and signal-dependent threshold (0) activation of the promoter (P). c. Raw CFP fluorescence (a.u.) of four single-cell hybrid oscillators over 40 hours (left panel). Filled diamonds denote expression peaks (of smoothed trajectories), for comparing oscillation timing between cells. Median trough, peak fluorescence: 2.0 a.u., 10.9 a.u., respectively. Power spectra (right panel) of the (mean-subtracted) trajectories exhibit a common peak frequency around 0.005 min⁻¹. d. Biological oscillators can be coupled by transporting a signal, S, across cell boundaries (top). The hybrid oscillators can similarly distribute a virtual signal between cells by multiplying their vector of signals, S, with a digitally-specified transfer matrix, T, at each time step. e. CFP trajectories of four coupled hybrid oscillators (left schematic and middle panel; 0.2 S is shared to the two nearest neighbours, per time step). Coincidence of expression peaks (filled diamonds) and near zero phase difference (right panel) indicate synchrony of the oscillators. Median trough, peak fluorescence: 1.8, 13.6 a.u. f-g. CFP trajectories (middle panel; filled diamonds: expression peaks) and phase difference (right panel) of 4-member (f) negatively-coupled (Median trough, peak fluorescence: 3.2, 18.9 a.u.), and (g) asymmetrically-coupled (Median trough, peak fluorescence: 1.5, 16.3 a.u.) groups of hybrid oscillators (+/−0.2 S transferred per time step).

FIG. 6

a. Scheme of the structure of the oscillator model M2. The model describes a network of an autonomously synchronizing population of synthetic oscillators through intercellular communication with a small signaling molecule, the autoinducer (red circle). The autoinducer is produced by LuxI, detected by LuxR, and can freely diffuse through the cell membrane. Solid black arrows: Transcription and translation. Dotted black and red arrows: Transcriptional activation or repression. Dash-dotted black arrows: Production of the autoinducer, diffusion of the autoinducer through the cell membrane and dimerization of the autoinducer with the LuxR receptor protein. b. Partitioning of the whole network into two separate modules, a communication and an oscillatory module (see text). Yellow thunderbolt: Exchange of the promoter of the respective gene with a light inducible transcription unit. Orange wiggly line: The respective protein is tagged with a fluorescence protein for detection using light microscopy. Orange circle (FP): Fluorescence protein under the transcriptional control of the LuxR-autoinducer complex.

FIG. 7.

Evaluation of the light inducible system (A), of an individual unit (B) and of a meta-unit composed of two individual units (C) for reconstructing the Respressilator described in Example 7. Each plot corresponds to a different experiment using a different light sequence to activate/deactivate the light inducible system. Dashed lines correspond to light inputs, while solid lines correspond to the respective median response over many individual replicas tested in parallel, quantified by the fluorescence output.

### DETAILED DESCRIPTION OF THE INVENTION

### Biological Circuits

The invention provides systems and methods for engineering, testing or modelling a biological circuit. The term “biological circuit” may in some cases be used interchangeably with the terms “biomolecular network” or “signalling network”. Typically the biological circuit is synthetic and may be rationally designed to perform an intended function. In other words the whole circuit does not occur naturally, although particular components or parts of the circuit comprising multiple components may be naturally occurring, or have been transferred from a different strain or species.

A biological circuit comprises multiple components that may interact with one another to control and define diverse cellular processes or behaviours. Typical components of a biological circuit include genes, promoters (inducible or constitutive), other regulatory elements such as enhancers, repressors, activators, gene products including sense and antisense RNAs, microRNAs, proteins, metabolites, sensors, bioproducts, ribosome binding sites, terminators, receptors, ligands, biorecognition molecules, biosensors (comprising (a) a biorecognition element that is capable of recognising a target molecule and (b) a physiochemical detector element such as an electrode capable of detecting a reaction caused by the recognition of the target molecule by the biorecognition element), reporters, transcellular communication molecules and enzymes. Other example components are those included in the Database of Standard Biological Parts. Furthermore, a previously designed and/or implemented circuit or a part of it can act as a component (sub-circuit) of a larger biological circuit in which it is integrated, allowing for modular composition of circuits. A biological circuit may comprise any combination of these components or classes of components or sub-circuits. In some cases a biological circuit may also interact with, respond to or influence elements of the internal or external environment of the cell or the cellular mileu, such as pH, temperature, metabolism, cellular survival and proliferation pathways, the cell cycle, and similar.

Biological circuits have applications in many different medical, industrial and environmental fields, including use, for example, as biosensors (e.g. to detect drugs/illegal substances, test water quality, detect bioweapons, detect/identify diseases based on human excrements and similar), in bio-materials/production (e.g. cobweb-like materials and similar), bio-computing, bio-reactors (producing, for example biofuels, enzymes such as enzymes use in or as washing agents, or pharmaceuticals), pollution management (e.g. in organisms used as part of wastewater treatment plants), in agriculture or animal/fish farms (e.g. for “smart” defense mechanisms against vermins, or to optimize fish and animal growth), in the development or implementation of lab-on-a-chip/organ-on-a-chip technology, and similar.

Examples of simple biological circuits that have been designed and/or fully implemented in living cells include logical gates, toggle-switches, oscillators, repressilators, counters and various types of sensors. In some cases in accordance with the present invention the biological circuit comprises or consists of one or more of these types of circuits.

### Living Cells

The systems and methods of the invention use one or more living cells. Any number of cells may be used, such 1 or more, 2 or more, 5 or more, 10 or more, 100 or more, 1000 or more, 10⁴ or more, 10⁵ or more, 10⁶ or more, 10⁷ or more, 10⁸ or more, 10⁹ or more, or 10¹⁰ or more cells.

The cells may be in vitro. The one or more cells may be present in an in vitro culture. In other cases the cells may be in vivo, for example in situ in an experimental model organism such as Zebrafish (Danio rerio), Caenorhabditis elegans, or Dropsophila, or other small invertebrate suitable for observing under a microscope. In other cases the cells may be in a tissue sample or synthetic organ.

The cells may be present in a culture flask or the wells of a flat plate, such as a standard 96 or 384 well plate, or in microfluidic channels. Such plates are commercially available from Fisher scientific, VWR, Nunc, Starstedt or Falcon. The culture may be present in a microfluidic device, such as the CellASIC ONIX Microfluidic Platform from Merck, the mother machine device. In other cases the cells may be present in a microfluidic designed for tissues or “organs”, such as those described by Frey, Olivier, et al. “Reconfigurable microfluidic hanging drop network for multi-tissue interaction and analysis.” Nature communications 5 (2014).

The flask, wells, device or other culturing means may be modified to facilitate culture of the cells, for instance by including a growth matrix. The flask or wells may be modified to allow attachment and immobilization of the one or more cells to the flask or wells. The surface(s) of the flask, wells, device or other culturing means may be coated with Fc receptors, capture antibodies, avidin:biotin, lectins, polymers or any other capture chemicals that bind to the one or more cells and immobilize or capture them.

The culturing means may include means for automatically or continuously supplying fresh media, optionally comprising chemical perturbations, and/or removing waste and/or excess cells. One or more of these functions may be controlled by the computer.

Typically the cells are undergoing cell division. In some cases continued growth of each cell is evaluated and cells that stop growing or die may be removed, for example automatically by the computer, from the biological circuit or experiment. In some cases it is preferable to start with a low initial number or concentration of cells so that the cells can be observed over a longer period of time. The culturing means typically permit long-term observation of individual cells or groups of cells. In some cases individual cells or groups of cells, or a majority of such cells or groups of cells used (for example 99%, 98%, 95%, 90%, or 80%) can be observed and/or a proliferation phenotype or growth and/or less than 100% or less than 90%, or 80% or 70% or 60% or 50% or 40% or 30% or 20% or 10% confluence maintained for at least 30 minutes, or at least 1 hour or 2, or 3, or 4, or 5, or 6, or 7, or 8, or 9,or 10, or 15, or 20, or 24, or 36, or 48, or 60, or 72, or 84, or 96 hours, or 1 week or 2 weeks or 3 weeks, or for at least 2 generations, or at least 3, or 4, or 5 or 10, or 20 or 30, or 40 or 50, or 100 generations.

In some cases individual cultured cells or groups of cells may be isolated from one another, for example in separate channels of a microfluidic device or in different microfluidic devices, or different wells or spots of a culture plate. Isolating the cells in this way may permit separate cell state measurements to be taken or differential control of the state or environment of different cells, as described further below. Isolating the cells may also permit intercellular communication between individual cells or groups of cells to be virtualised, as described further below, or for an experiment to be repeated multiple times in parallel under the same or divergent conditions.

The groups of cells may, for example, be clonal populations or groups containing only direct progeny. In other cases, different parts of the biological circuit may be implemented in different groups of cells. Such groups may be homogenous or may comprise further subgroups in which separate sub-parts of the biological circuit are implemented. Alternatively different isolated cells or groups of cells may replicate the same part of the biological circuit, under the same or different environmental conditions. In yet other cases, different groups might represent different cell types originating from the same organism, like differentiated and undifferentiated cells.

Conditions for culturing cells are known in the art and vary according to the cell, tissue or organism. A specific example is culture at 37° C., 5% CO₂ in medium supplemented with serum.

The one or more cells may be any type of cells. Suitable cells for use in the invention include prokaryotic cells and eukaryotic cells. The prokaryotic cell may be a bacterial cell. Suitable bacterial cells include, but are not limited to, Escherichia coli, Corynebacterium and Pseudomonas fluorescens. Suitable eukaryotic cells include, but are not limited to, Saccharomyces cerevisiae, Pichia pastoris, filamentous fungi, such as Aspergillus, Trichoderma and Myceliophthora thermophila C1, baculovirus-infected insect cells, such as Sf19, Sf21 and High Five strains, non-lytic insect cells, Leishmania cells, plant cells, such as tobacco plant cells, and mammalian cells, such as Bos primigenius cells (Bovine), Mus musculus cells (Mouse), Chinese Hamster Ovary (CHO) cells, Human Embryonic Kidney (HEK) cells, Baby Hamster Kidney (BHK) cells and HeLa cells. Other example mammalian cells include, but are not limited to, PC12, HEK293, HEK293A, HEK293T, CHO, BHK-21, HeLa, ARPE-19, RAW264.7, M38K and COS cells. In general any cell line that is amenable to genetic manipulation may be used. In some cases, however, cells that have not been genetically manipulated could be used. In such cases the cellular and simulated parts of the biological circuit could interact through, for example, physical perturbations and/or through existing or naturally occurring sensory pathway(s).

Where a population of cells is used, the cells of the population may be homogenous. For example a clonal microbial population may be used. Alternatively a heterogenous population of cells including different cell types, strains or species may be used. In some cases the cellular part of the biological circuit implemented in the cell population will be the same in each cell of the population. In other cases different parts of the biological circuit may be implemented in different cells or cell types, strains or species.

### Means for Controlling the Cell State or Environment

In some embodiments, the means for controlling the cell state or environment comprises means for emitting light. The system may make use of optogenetics, using light, optionally light of a specific and/or different intensities or wavelengths, as a means for regulating or controlling a light-sensitive element or component of the system, such as a light sensitive protein.

For example, in some embodiments the cellular part of the biological circuit may comprise one or more light inducible or regulatable transcriptional activators or light-switchable/inducible promoters for controlling transcriptional activation of one or more genes or other light-responsive elements. A light-regulated promoter may be light-inducible or light-repressible. An example is a system or method using the light-switchable gene promoter system developed by Shimizu-Sato et al “A light switchable gene promoter system” Nature Biotechnology, vol. 20, pp. 1041-1044, (2002) and Mendelsohn “An enlightened genetic switch” Nature Biotechnology, vol. 20, pp. 985-987, (2002), or a modified version thereof. This system is based on phytochrome phyB, a holoprotein that is mainly responsible for regulating plant growth in response to environmental light signals. To be light sensitive, phyB has to be linked to tetrapyrrole chromophore, a molecule which must be provided for the light switchable promoter system by an external source or, alternatively, be produced inside the cell by the introduction of additional genes. The holoprotein has two forms, Pr and Pfr, the latter being the biologically active form. Transitions between the Pr and the Pfr form can be stimulated by red light and vice versa by far-red light. The active form can interact with another protein, PIF3, but the inactive form Pr cannot. Shimizu-Sato et al. fused the photosensory N-terminal domain of phyB to the GAL4 DNA-binding domain (phyB-GBD) and PIF3 to the GAL4 activation domain (PIF3-GAD). The new synthetic protein phyB-GBD can bind to its DNA binding site Gal4 UAS, but only activates transcription in its active form Pfr when it can form a complex with the synthetic protein PIF3-GAD. Upon activation with red light, the gene with the Gal4 UAS promoter is transcribed constitutively until deactivation of the transcription with far-red light. The transition between the minimal and maximal transcription rates is reported to be fast. Furthermore, the amount of activation of phyB can be precisely controlled by regulating the amount of photons used to activate or deactivate the holoproteins.

Other examples of light-responsive elements include those that regulate expression of the small subunit of ribulose-1,5-bisphosphate carboxylase-oxygenase (rbcS) gene, the chlorophyl a/b binding protein, and the chalcone synthase.

Other examples of systems or components that can be controlled by exposure to light include light-sensitive ion-channels or a light-inducible translocation system, for example the system of Levskaya, Anselm, et al. “Spatiotemporal control of cell signalling using a light-switchable protein interaction.” Nature 461.7266 (2009): 997. Another example is a light-inducible degradation system, which may be used to regulate or control the concentration of a light-sensitive element or components such as a light-sensitive protein. An example of such a system is that described in Tyszkiewicz and Mir, “Activation of protein splicing with light in yeast,” Nature Methods, vol 5, no. 4, pp. 303-305 (2008).

In some embodiments, the means for controlling the cell state or environment comprises means for exposing the cell(s) to changes in temperature, pH, or air or oxygen levels/anaeobiosis, or to water or salt stress, or means for wounding the cell(s). In some embodiments, the means for controlling the cell state or environment comprises means for exposing the cell(s) to one or more chemical modulators, such as a chemical inducer. Examples are antibiotics (such as tetracycline), alcohols (such as the alcohol dehydrogenase gene promoter), steroids, metals, pheromones, metabolites and small molecules such as sugars (such as lactose), salicylic acid, ethylene or benzothiadiazole. An example is the system described in Ottoz, Diana S M, Fabian Rudolf, and Jörg Stelling. “Inducible, tightly regulated and growth condition-independent transcription factor in Saccharomyces cerevisiae.” Nucleic acids research 42.17 (2014): e130-e130.

In some cases the general conditions in which the cellular part of the biological circuit is to operate are controlled, and may be experimentally defined or determined by the output from the simulated part of the biological circuit. In some cases the imposed conditions may influence the behaviour of the cellular part of the biological circuit without requiring any specific engineering of the cells to be responsive. In other cases, the biological circuit may comprise or have been engineered to comprise one or more specific components that are controlled by environmental factors, such as a light-, temperature-(heat-shock or cold-shock) or chemically-regulated promoter, or similar regulatory elements controlled by any one or more of the factors discussed above. In some cases the biological circuit makes use of a naturally occurring pathway that has been rewired to provide for input into the cellular part of the biological circuit. An example is the system described in Park, Sang-Hyun, Ali Zarrinpar, and Wendell A. Lim. “Rewiring MAP kinase pathways using alternative scaffold assembly mechanisms.” Science 299.5609 (2003): 1061-1064.

In some cases in accordance with the present invention the state or environment of different cells in a population of cells (in which the cellular part of the biological system may be implemented) may be differentially controlled.

In some aspects, the present invention provides a method of restraining variability in a cell population, or of programming a cell population to maintain a specified static or dynamic behavioural distribution, such as in the expression of a gene. The method may comprise taking measurements of a cell state parameter, such as gene expression, from individual cells in the population and providing feedback control at the single cell level.

### Means for Taking Measurements of One or More Cell State or Environmental Parameters

Any suitable means may be used for taking measurements of one or more cell state or environmental parameters from the one or more living cells in accordance with the present invention.

In some cases the expression by the cells of one or more genes of interest is measured. Typically one or more copies of the gene of interest in the cells is replaced with a reporter gene, such as a fluorescent protein, or with a modified version of the gene that incorporates a reporter, for example a fluorescent tag. The replacement gene is typically under the same genetic control as the original gene of interest. In other cases the expression or cellular level of a component of interest is measured by using it as a regulator of the expression of a reporter gene, such as for a fluorescent protein. In either case, expression of the reporter is measured and an estimate of the corresponding levels of expression of the original gene or component of interest is calculated. Preferably the fluorescent reporter has a fast maturation time, such as less than 30 minutes, or less than 40 or 50, or 60, or 70, or 80 or 90 minutes.

The simulated part of the biological circuit is simulated in real time by the computer, in the sense that it exchanges inputs and outputs with living cells in a timeframe that is meaningful for modelling, testing or engineering a biological circuit that is split between a cellular part implemented in the cells and the simulated part. In some cases a measurement may be taken and/or input to the cellular part of the circuit via the controlling means is provided continuously. In other cases a measurement is taken and/or input to the cellular part is provided periodically. The measurements may be quantitative or may comprise the detection of a change in the level or frequency of a parameter. The data collected from the measurements may be automatically processed by the computer to provide the input into the simulated part of the biological circuit. When a measurement is taken or input provided periodically, a suitable frequency will depend on the timeframe over which relevant cellular processes operate. In some cases the mechanism used to take a measurement and/or provide an input may introduce a delay. In some cases this can be factored into the simulation and/or allowed for in setting the computer-controlled input into the cellular part of the biological circuit. For example, the maturation times of even the fastest available fluorescence proteins may, for some species, be above twenty minutes, so measured fluorescence typically indicates the cellular state as it was in the past. This delay may be bypassed by estimating the real time cell state/parameter(s) based on already measured (fluorescence) outputs and the known (light) inputs. A fluorescence microscope may be used. For example a fluorescent microscope with motorized x y and z control allows appropriate measurements to be taken from different cells or groups of cells individually. Light-emitting diode arrays may be installed as light sources, for example for red light (660 nm) and far-red light (748 nm) pulses. In some embodiments, the microscope may be connected to a work station using the core drivers and interfaces of μManager (see http://www.micro-manager.org) for control of automated microscopes. To provide a mechanism to change the cell's input signal depending on its fluorescence the open source microscope software YouScope (www.youscope.org, Lang, M., Rudolf, F., & Stelling, J. (2012) Use of YouScope to Implement Systematic Microscopy Protocols. Current Protocols in Molecular Biology, 14-21) may be used, which enables for parallel acquisition of images and the modification of light input signals separately to different cells or groups of cells, for example for each well of a microplate. YouScope uses the Java interface of the μManager core to control the microscope and can be configured by a separate platform-independent visual user interface. YouScope allows scripting of the complete or parts of the measurement protocol with Matlab (The Math Works, Natick, Mass.), Jython, JavaScript and similar.For example, a Matlab script may be executed regularly by YouScope for every group of cells/well in the microplate, which triggers the microscope to take an out-of-focus and a fluorescence image with a respective filter. Afterwards, the script invokes the segmentation software CellX (Mayer C, Dimopoulos S, Rudolf F, Stelling J (2013) “Using CellX to quantify intracellular events” Curr Protoc Mol Biol Chapter 14: Unit 14 22, or Dimopoulos S, Mayer CE, Rudolf F, Stelling J (2014) “Accurate cell segmentation in microscopy images using membrane patterns” Bioinformatics 30: 2644-2651) to detect and track the cells, and to estimate their fluorescence signal. In one example, for every well, one cell is selected and its fluorescence signal is used as the input signal for the real time simulation of the in silico part of the biological network using the ODE solver ode15s of Matlab. Based on the output, the Matlab script triggers either a red light (660 nm), a far-red light (748 nm) or no pulse in the respective well. The images made, the estimated cell positions and properties, the estimated fluorescence signal and the applied light impulse are stored for every well for later analysis.

In some cases the presence or concentration of one or more metabolites or molecule produced and optionally secreted or excreted by the cells is measured. Such molecules might include nucleic acids, (for example DNA, mRNA, microRNA, and small interfering RNAs), proteins, antibodies, receptors, ligands, signalling molecules, protein complexes and toxins. For example, Bacchus et al. “Synthetic two-way communication between mammalian cells.” Nature biotechnology 30.10 (2012): 991-996 describes synthetic conversion of indole to tryptophan, and acetaldehyde to ethanol, for which commercial essays are available. The same paper also describes detection, using standard assays, of human placental secreted alkaline phosphatase (SEAP) and secreted alpha-amylase (SAMY) secreted into the medium by engineered mammalian cells as an output indicative of cell state Another technology would be to connect the medium outflow to a real-time mass spectrometry to quantify most of the metabolites in real time, as done in Hold et al., “Forward design of a complex enzyme cascade reaction.” Nature communications 7 (2016).

Cell state parameters that may be measured include cell growth, cell division, reproduction, rate of cell growth, division, or reproduction, cell number, cell density, cell confluence, viability, respiration, cell morphology, cell shape, cell adhesion, spatial organisation of tissues, metabolic condition, cell motility, cell movement, cytoskeletal arrangement, cytoplasmic movement, intracellular trafficking, electrophysiological state, firing times of neurons, degree of differentiation, expression of specific molecules such as ligands or receptors on the cell surface, receptor activation, pH and temperature.

In some case the operation of the cellular part of the biological circuit in the living cells influences measurable parameters of the environment of the cells that may be measured as an output of the cellular part of the biological circuit, or of a part of the biological circuit operating in a particular cell or group of cells. Environmental parameters that may be measured include pH, temperature, light or fluorescence emission or wavelength frequency, oxygen saturation, or the presence or concentration of any secreted or excreted molecules or metabolites as described above.

### Interactions Between Cells

In some cases in accordance with the invention, the biological circuit operates in a single bio-digital hybrid cell. A bio-digital hybrid cell comprises a living cell in which a cellular part of a biological circuit is implemented, and a virtual counterpart within the simulated part of the biological circuit, wherein the virtual counterpart is a simulation of a part of the biological circuit as it would operate if it were implemented in the counterpart living cell. In the case of a single cell biological circuit, the system or method of the invention tests, models or predicts how the biological circuit would operate if the simulated part were additionally implemented in the cell, in other words if the whole biological circuit was implemented in a single cell.

In some cases the system or method of the invention may be used to engineer, test or model a population of cells. The population may be virtual in the sense that that the different cells or groups of cells of the population are physically isolated from each other and the cellular part of the biological circuit implemented in different cells do not directly interact, other than optionally via the simulated part of the biological circuit as described further below.

In some cases the system or method of the invention is used to engineer, test or model a multicellular biological circuit, in which different parts of the biological circuit operate in different living, virtual, and/or bio-digital hybrid cells and interact with one another. The interaction may operate (i) wholly in the cellular part of the biological circuit, for example between living cells that are cultured together; (ii) wholly in the simulated part of the biological circuit, e.g. between virtual counterparts of living cells; or (iii) partly in the cellular part and partly in the simulated part of the biological circuit. The simulated part of the biological circuit may also include additional, wholly virtual cells which may virtually interact with bio-hybrid cells/virtual counterparts and optionally with each other.

In some cases in accordance with the invention a population of cells is cultured and the means for controlling the state or environment of the living cells is set based on measurements of one or more cell state or environmental parameters taken from one or more other cells in the population of living cells. For example this may be the case when simulated parts of the biological circuit that operate in virtual counterparts of living cells virtually interact with each other in the simulation. In this case the cell state or environmental parameter measurements taken from the living cells provide input into the simulated parts operating in the virtual counterparts of the living cells. This in turn may affect the interaction between the simulated parts operating in the different virtual counterparts, and subsequently the input from the simulation back to different living cells.

In other cases, there are no individual bio-digital hybrid cells or specific virtual counterparts to the living cells within the simulation. Instead the input into the simulated part of the biological circuit may be provided by, for example, averaged or otherwise combined cell state or environmental measurements taken from multiple living cells (for example the average emitted fluorescence), measurements taken from a random or representative sample of cells selected from the population, or from environmental parameter measurements to which multiple living cells contribute (for example the concentration of a metabolite produced by the cells and secreted into the culture media). In this case the means for controlling the state or environment of the cells may also be set based on measurements of one or more cell state or environmental parameters taken from one or more other cells in the population.

In some cases multiple experiments can be performed in parallel using the same system, method or experimental set-up. The different experiments may test or model different biological circuits. Alternatively, the different experiments may test or model duplicates of the same biological circuit split in the same way between the cellular and simulated parts. Different experiments may also be carried out under different environmental conditions, for example different temperature, pH, cell density or culture medium, or with different cell types, strains or species. The simulations from each of the experiments may be run simultaneously on the same computer. The outcome of different experiment can be compared to assess the variation introduced either by natural differences between different cells or groups of cells, or by differences in the biological circuits, environmental conditions or cell types, strain or species.

In some cases specific intercellular communication between different bio-digital hybrid cells may be virtualised via the simulated part of the biological circuit. The digital communication between individual bio-digital hybrid cells may be freely-specifiable. In other words, the simulation controlled by the computer specifies which hybrid bio-digital cells interact with each other, and the nature of the interaction.

For example, in one embodiment, the simulation and/or computer specifies that the part of the biological circuit that operates in a first bio-digital hybrid cell interacts in a specified way with one or more other specific bio-digital hybrid cells. In some cases the one or more other specific bio-digital hybrid cells may be considered virtual physical neighbours of the first bio-digital hybrid cell. A selected output from the part of the biological circuit that operates in the first bio-digital hybrid cell may be shared between the one or more other specific bio-digital hybrid cells in a specified way. For instance, in a simple example a particular output from the first bio-digital hybrid cell may be shared between the first bio-digital hybrid cell and one other specific bio-digital hybrid cell, or may be shared between two other specific bio-digital hybrid cells, or three or four or five or six or seven or any specified number of other cells and optionally also with the first bio-digital hybrid cell. The shares may be equal or may have a different distribution specified by the simulation and/or controlled by the computer.

In a simple example a cell state or environmental parameter measurement taken from a first living cell is processed by the computer and directly fed back to one or more other specific living cells of the population of cells in which the biological circuit operates via means for differentially controlling the state or environment of different cells in the population. In other cases, one or more cell state or environmental parameter measurements taken from a first living cell provides input into the simulated part of the biological circuit that operates in a virtual counterpart to the first living cell. Output from that simulated part then provides input to one or more other specific living cells of the population of cells in which the biological circuit operates via means for differentially controlling the state or environment of different cells in the population. In either case, the virtual communication between specified individual cells may be repeated across all or a subpopulation of the cells in the population in which the biological circuit operates.

In some cases the living cells corresponding to the hybrid bio-digital cells are physically isolated from each other and only interact via the virtual connections controlled by the simulated part of the biological circuit. In other cases the virtual neighbouring cells may also be physical neighbours and the cellular parts of the biological circuit that are implemented in the physically neighbouring living cells may additionally interact with each other.

### Emergent Behaviour

In some cases the systems and methods of the present invention may be used to predict or analyse emergent behaviour in a population of cells in which a biological circuit or part of a biological circuit is implemented. Emergent behaviour describes the global consequence of interactions between individual cells (living, virtual and/or bio-digital hybrid) in the population of living, virtual and/or bio-digital hybrid cells.

In some cases, some or all of the living cells may be exposed to a chemical or environmental perturbation, such as the introduction of an antibiotic or toxin, or a nutrient, pH or temperature shift. In other cases elements of the simulated part of the biological circuit can be perturbed. This can be particularly useful in providing information about the robustness of the cellular part of the biological system and its sensitivity to changes in the simulated part of the biological circuit.

In some cases, emergent population behaviours can originate from differential behaviour in one or more individual cells. Accordingly in some cases the method of the present invention comprises introducing into the cell population one or more living, virtual, or bio-digital hybrid cells that has divergent behaviour from the other living, virtual and/or bio-digital hybrid cells of the population, and/or in which a part of the biological circuit operates in the mutant cell(s) and is different from that implemented in or simulated for other, non-mutant cells of the population. The introduced cell(s) may be referred to as “mutant” cells. The introduction of the mutant cells may perturb or alter the behaviour of the other cells in the population or perturb or alter the operation of other parts of the biological circuit.

### Iterative Biological Circuit Design Cycles

At present, one of the most advanced ways to construct novel biological signalling networks in synthetic biology is using iterative rational design or iterative engineering. This can be described as an iterative cycle, which consists of four steps: First, a model of the intended network is constructed. Second, the model is analyzed and modified (where appropriate) to find a promising network design. Third, this design is implemented in a genetic circuit, and fourth, its properties and dynamic behaviour are determined by experiments. Since the predictive power of models of signalling pathways are still relatively low compared to other engineering disciplines, it is likely that the initial network design will not result in a sufficiently good biological implementation. Thus the existing model has to be refined according to the experimental results. The iterative cycle is repeated until a satisfying implementation is obtained.

Since the genetic implementation of a synthetic network is time consuming and costly, and the number of necessary iterations through the rational design cycle in general increases with network size, most synthetic networks already implemented are relatively small and can only achieve simple tasks.

The inventors propose dividing a synthetic network into smaller and simpler subnetworks (denoted as modules) and testing the functionality of each of these subnetworks separately in an inner, smaller iterative rational design cycle. If the network size is large, several of the modules can afterwards be merged to second or third order modules and tested in their own iterative circles. Since the single modules are smaller and simpler than the overall network, only few iterations are needed in every inner cycle. Furthermore, the construction and validation of the modules are independent of each other, so that they can be done in parallel at the same time and even by different experimenters. After the validation and—if necessary—modification of each module the complete network is merged and experimentally validated in an outer rational design circle. The present invention provides systems and methods that enable testing of the dynamic behaviour of subnetworks within a biological circuit to be tested in their natural environment.

Two submodels may be extracted from the overall model of the synthetic network, optionally prior to the genetic implementation of a module. One consists of the dynamics of the subnetwork (M+) and the other consisting of the whole network except the subnetwork which should be implemented and tested (M−). The subnetwork M+ is implemented in one or more living cells and may be modified or adapted so that relevant inputs can be fed in and outputs can be measured. The outputs of M− are the inputs for M+ and vice versa.

Using this approach, one is able to validate the correct dynamic functionality of a single biological module (the cellular part of the biological circuit) under the assumption that all other modules (the remaining simulated part of the biological circuit) work as predicted by the overall model. By repeating this approach, one is furthermore able to obtain statistics on the reliability of these results and their dependency on the cell size, the cell cycle, cellular noise, and other physiological parameters which might or might not influence the performance of a given module.

Accordingly in some cases the method of the present invention comprises engineering, testing or modelling a first biological circuit according to any of the methods of the invention described above, optionally modifying the cellular part and/or the simulated part of the first biological circuit, and further engineering, testing or modelling a second biological circuit according to any of the methods of the invention described above, wherein the second modified biological circuit is a modified version of the first biological circuit. In some cases the modification comprises implementing in the cellular part of the second biological circuit an element that was simulated by the computer in the first biological circuit. In other cases the modification comprises simulating in the computer an element of the second biological circuit that was implemented in the living cell(s) in the first biological circuit.

### EXAMPLES

### Introduction—Examples 1-5

Predicting the behaviour of individual bacteria and bacterial populations is challenging and the complexity of the task increases rapidly already in the simplest laboratory conditions that include population heterogeneity and ecological or environmental interactions. Even clonal groups of microbes can interact with each other and with nearby organisms, undergo spatial and functional organization, insulate their populations from transient stresses, including antibiotics, and coordinate virulence. Therefore, to understand and manipulate natural or engineered bacterial populations, we require the ability to experimentally measure and control factors in individual cells that generate emergent population behaviours.

Recent technological advances have facilitated experiments at the single cell level in defined conditions. Microfluidic devices enable long-term observation of individual cells and precise environmental control. However, differentially perturbing many individual cells is technically involved. Molecular genetics techniques permit straightforward design of synthetic genetic circuits to assay their effects at the population level. However, in vivo behaviour of even simple synthetic circuits is often hard to predict, and disentangling interactions between their components and with the host remains a laborious task.

To this end, we constructed a general purpose, automated platform to programmatically measure and control gene expression in lots of individual bacterial cells over many generations, while dynamically modulating the chemical environment of the cells. The platform we developed combines microfluidics and optogenetics and enables simultaneous, quantifiable light-responsive control of gene expression over several days in hundreds of individual bacteria, as well as global chemical perturbation (e.g. nutrient shifts, toxin exposure). The platform is run by a computer that defines and controls the entire experiment, analyzes the data online, and uses independent software controllers to automatically adjust scheduled light perturbation sequences on the fly for each individual bacterium.

### Example 1—Population Structuring by Independent Closed-Loop Control of Gene Expression in Many Individual Cells

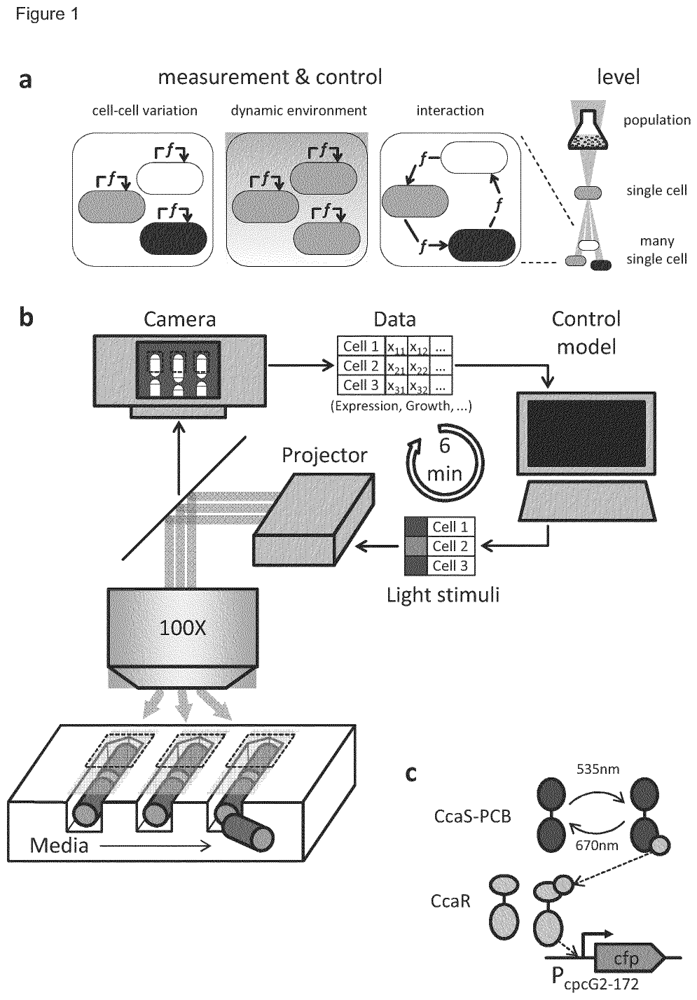

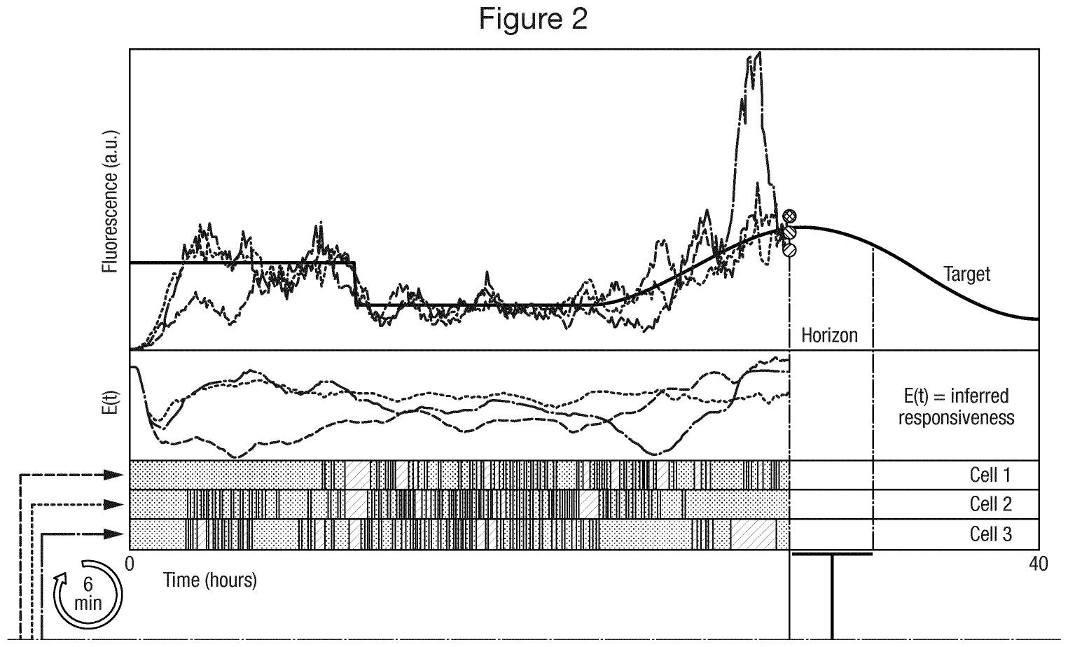

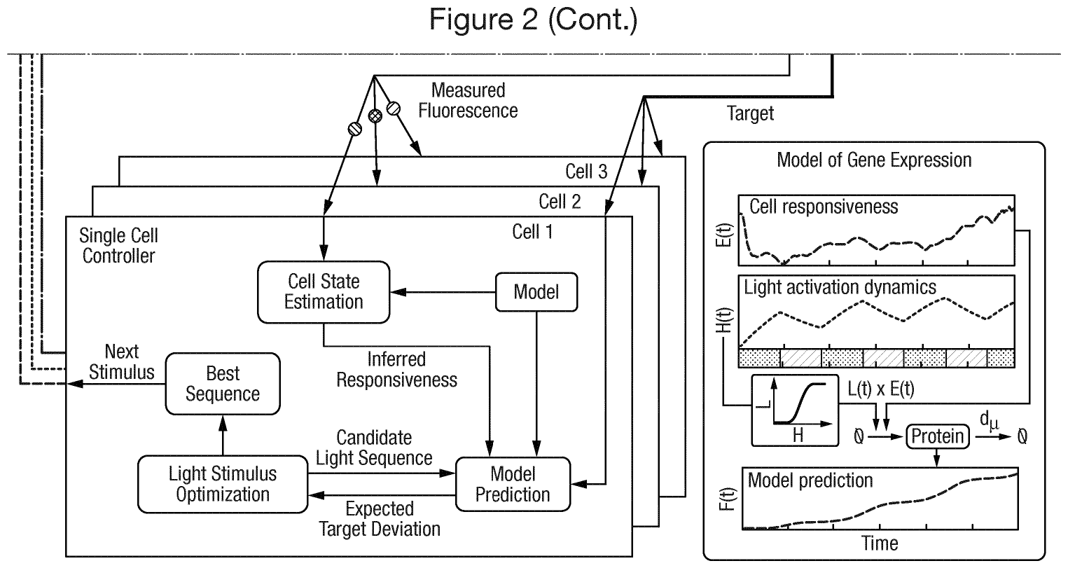

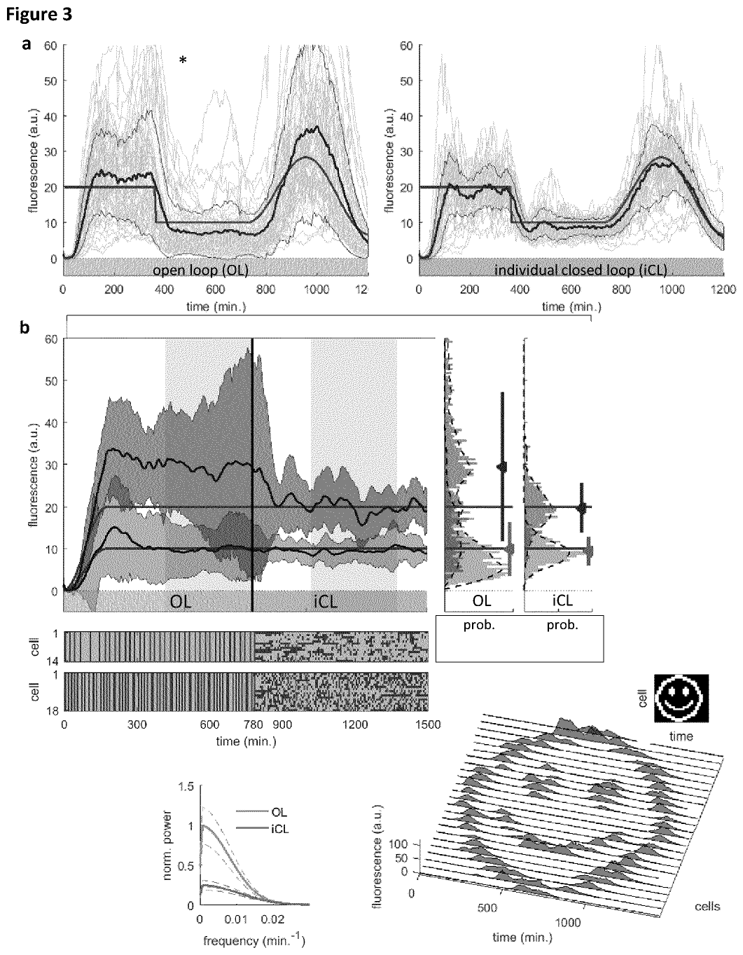

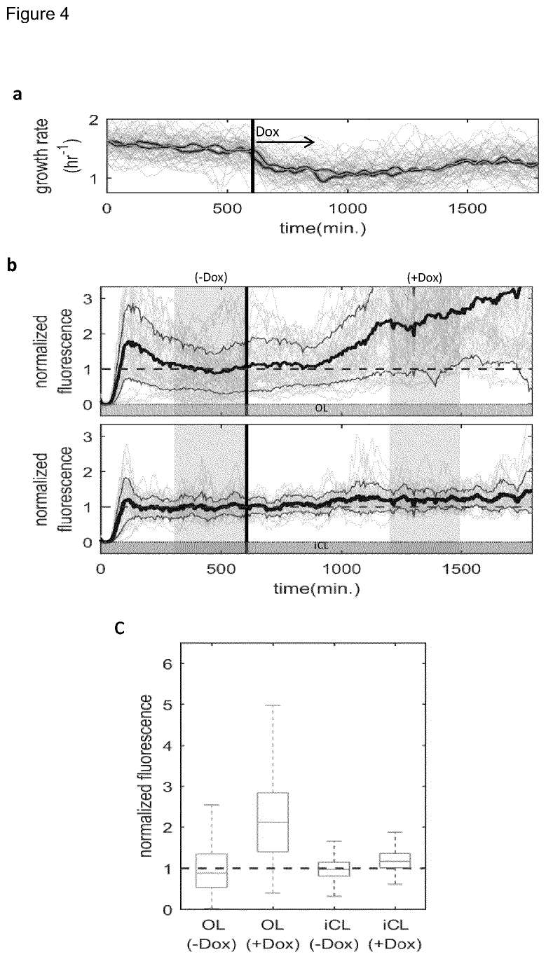

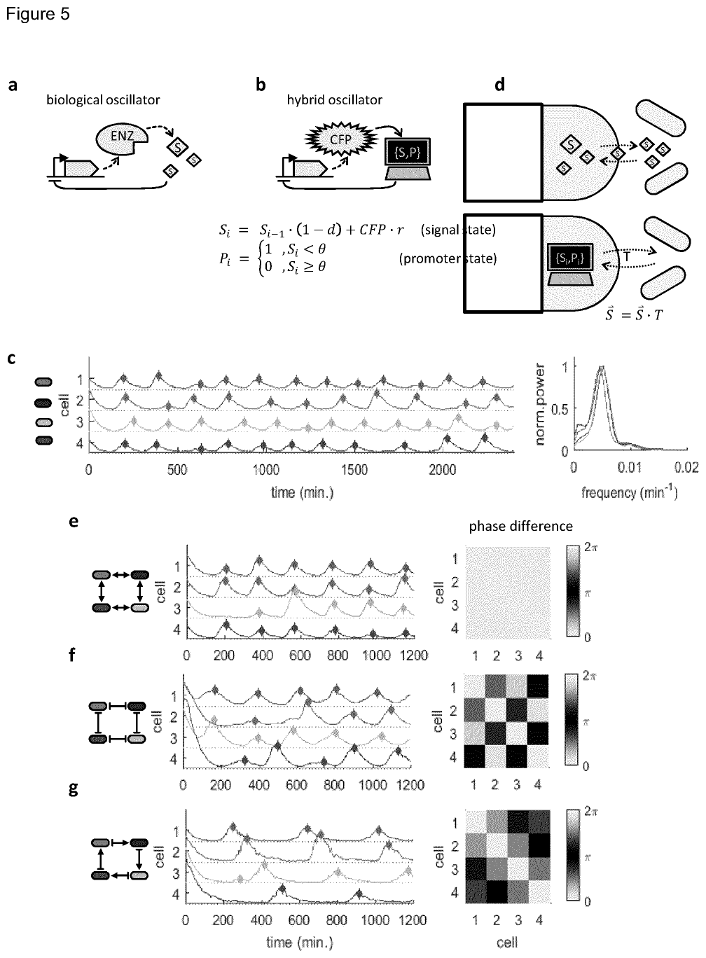

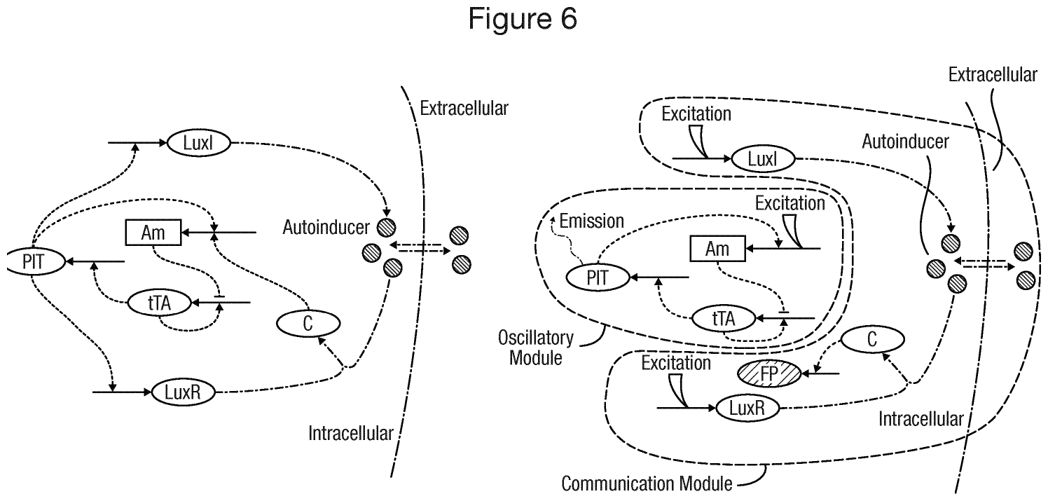

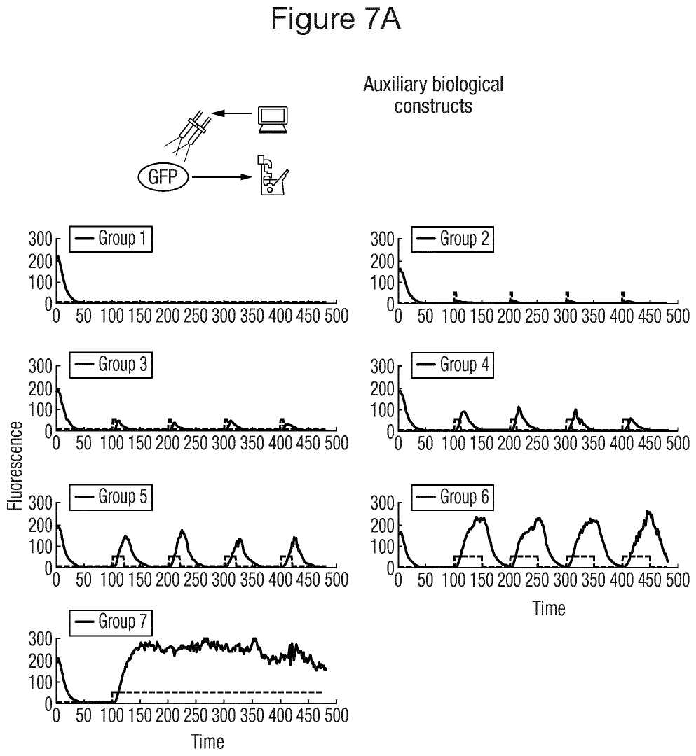

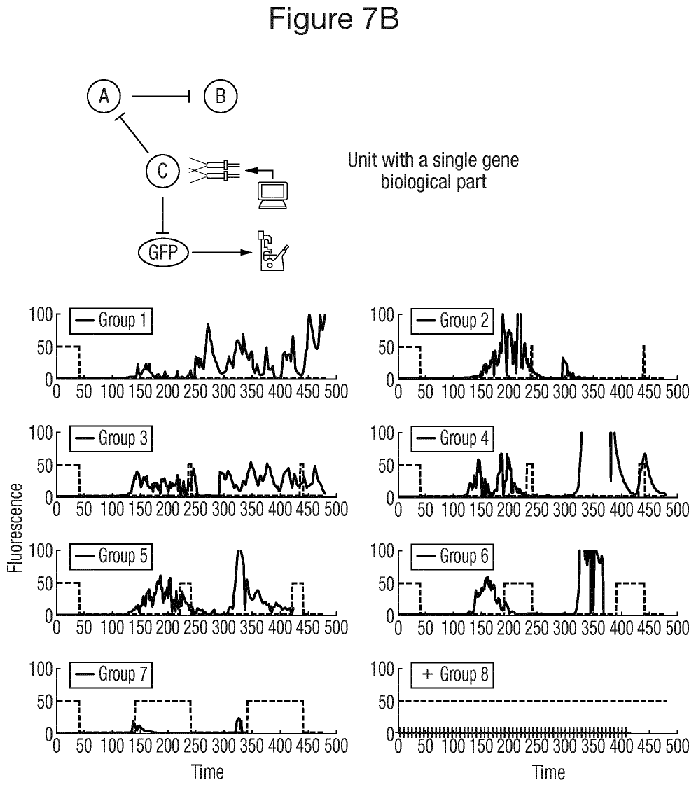

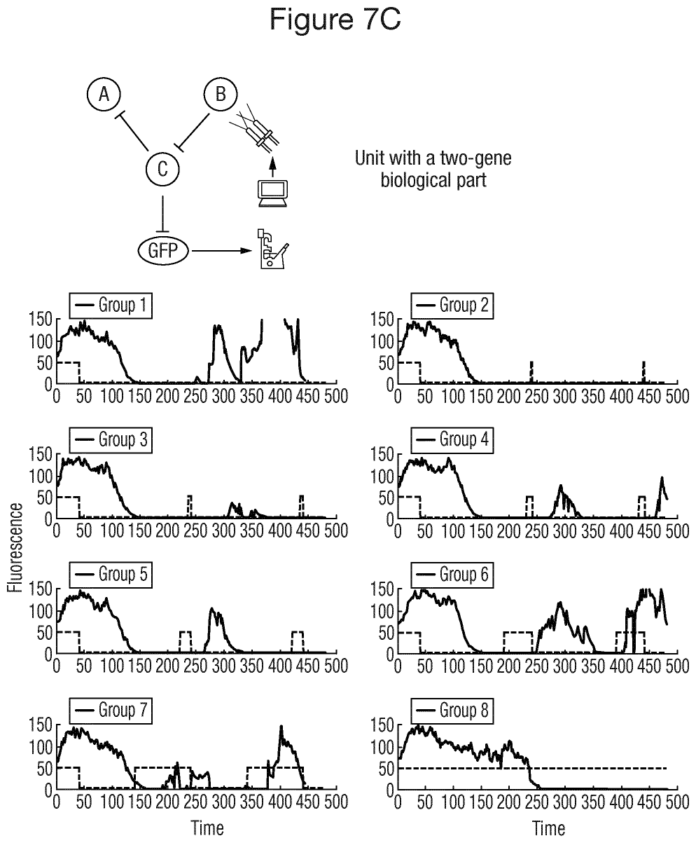

Here we describe a general purpose, automated platform to programmatically measure and control gene expression in lots of individual bacterial cells over many generations, while dynamically modulating the chemical environment of the cells. The platform combines microfluidics, image-based gene expression and growth measurements, and on-line optogenetic expression control, and enables simultaneous, quantifiable light-responsive control of gene expression over several days in hundreds of individual bacteria, as well as global chemical perturbation (e.g. nutrient shifts, toxin exposure). The platform is run by a computer that defines and controls the entire experiment, analyzes the data online, and uses independent software controllers to automatically adjust scheduled light perturbation sequences on the fly for each individual bacterium (FIG. 1a).

### Experimental Setup

We constructed the setup outlined above to perform a measurement-and-control loop (FIG. 1b, Methods) on E. coli cells bearing a light-regulated gene transcription module. In this setup, we grow and track individual cells over hours or days, confined at closed ends of short (˜23 um) cell-width channels in a microfluidic “mother machine” device, on a temperature-controlled fluorescence microscope¹⁴. Larger channels intersect the growth channels, supplying fresh nutrient media and chemical perturbations and removing waste and excess cells. Gene expression is estimated using image intensities of a fluorescent reporter. Since reporter levels vary too much for reliable segmentation morphological cell data are acquired by imaging a second, constitutively-expressed, fluorescent reporter. Software controllers, associated with individual cells or cell groups, process these data and return expression activation/repression signals for delivery to each cell. Cells are individually stimulated by projecting an RGB image of the signal intensities, mapped to appropriate color channel and cell locations, onto the light-responsive cells using a modified overhead projector (Methods). Six minute control intervals permit tracking and control of 200-400 cells.

In closed-loop mode, once defined, experiments are implemented entirely programmatically and without intervention. To enable fully autonomous operation, we wrote custom MATLAB software to manage all aspects of the experiment (Methods). The modular software eases modifications of the physical system, and simplifies revision of controllers and experiment protocols.

To independently regulate expression in single cells, we rely on light-responsive transcription mechanisms. We employ a recently-optimized ccaSR-based system with low leak and large dynamic range, that includes a synthesis pathway for the necessary phycocyanobilin (PCB) chromophore (Schmidl, S. R., Sheth, R. U., Wu, A. & Tabor, J. J. Refactoring and Optimization of Light-Switchable Escherichia coli Two-Component Systems. ACS Synth. Biol. 3, 820-831 (2014). Illumination with green (˜535 nm) or red (˜670 nm) light respectively activates or deactivates expression from the P_{cpcG2-172} promoter with timescales on the order of several minutes(see Olson et al. “Characterizing bacterial gene circuit dynamics with optically programmed gene expression signals” Nat. Methods, advance online publication, (2014), Schmidl et al “Refactoring and optimization of light-switchable Escherichia coli Two-Component systems” ACS Synth. Biol, vol. 3, pp. 820-831, (2014) and Hirose et al. “Cyanobacteriochrome CcaS is the green light receptor that induces the expression of phycobilisome linker protein” Proc. Natl. Acad. Sci, vol. 105, pp. 9528-9533, (2008)). For testing our setup, we have placed a cyan fluorescent protein expression reporter, Cerulean (cfp), under the control of the P_{cpcG2-172} promoter (FIG. 1c).

### Single Cell Control

We verified our system by controlling gene expression patterns in a small population of Escherichia coli. In cell population control, mean and individual error represent two important deviations of expression from target levels, and depend broadly on the control type (Table 1).

TABLE 1
Error level reduction by control type

| Control Type | Mean Error Reduction | Individual Error Reduction |
|---|---|---|
| Open Loop (OL) | No | No |
| Population-level | Yes | No |
| Closed Loop (pCL) | | |
| Closed Loop | Yes | Yes |
| Closed Loop (iCL) | | |

Open loop (OL) controllers precompute light stimulation sequences based on an average cell response model. OL controllers suffer from both mean and individual error. Mismatches between modeled and actual population responses produce systematic errors in mean expression that are sensitive to unmodeled changes in cell behavior (e.g., with environment). Further, “average cell” stimuli fail to account for cell-to-cell differences in response, and cannot contain the resulting variation in expression. By comparing expected and measured responses to the control stimulus, population-level closed loop (pCL) controllers substantially reduce mean target deviations by adjusting global stimuli on-the-fly. However, stimuli applied uniformly across the population cannot contain variation stemming from differences between cells. To reduce such individual error, controllers should operate at the single cell level at which the error is generated. We tested whether closed loop control at the level of a single cells could be extending to parallel, individual-level closed loop (iCL) control of a population of many cells within our device could mitigate both sources of error.

To control gene expression in individual cells, we used a receding-horizon control scheme (FIG. 2) based on a simplistic (although predictive) stochastic kinetic model that we identified from several calibration experiments. The model incorporates an internal (unmeasured) state, hereafter termed “cell responsiveness” (FIG. 2) that can vary between cells and in time. Every six minutes, for each cell, the controller compares the recorded fluorescence level to a predicted level calculated from the model and updates its estimate about the cell's responsiveness by weighting prediction and measurement according to their uncertainties. Measurement uncertainty stems from technical errors in recording cells' fluorescence whereas prediction uncertainty is a consequence of stochasticity in modeled chemical reactions and the imperfectly known, possibly time-varying, cell responsiveness. The prediction uncertainty can be efficiently calculated from the stochastic model of the system using moment equations. The controller then uses the updated estimate of the cell's responsiveness to identify a light sequence that minimizes the deviation of the expected fluorescence levels in the cell from the desired target profile over a certain planning horizon (FIG. 2).

We tested this control scheme by making individual cells track a target fluorescence profile consisting of steps and a sine-wave. To quantify control performance, we split the cells into two groups. For the first group, we used OL control to pre-calculate the entire optimal light sequence (based on a mean population response model identified from calibration experiments) without online learning of the responsiveness or feedback of the cells. The second group was iCL feedback-controlled as described in FIG. 2 (see sample cells). The resulting mean fluorescence of the OL-controlled group of cells (FIG. 3a, left panel, blue line) is roughly correct, implying that our stochastic model is predictive over long-time horizons. However, the control performance is less satisfactory for individual cells (grey lines). Heterogeneity in a population exposed to an identical light stimulation pattern results in widely varying fluorescence trajectories across the cells and, thus, high variance around the mean response. In particular, fluorescence of some cells is either far above or far below the average for long periods of time. We expect individualized light patterns based on online learning of each cell's responsiveness to decrease stimulation of over-responsive cells and increase it for under-responsive cells, reducing both the mean error and expression heterogeneity of the population. Indeed, the iCL-controlled cells exhibit both a reduced error in mean fluorescence, and a narrower distribution around that mean than cells under OL control (FIG. 3a, right panel).

### Example 2—Defining Population Expression Distributions

The effectiveness of iCL control in reducing mean error and cell-to-cell variation in expression suggested that—beyond simply narrowing population expression around single targets—we could use such control to specify more complex distributions of gene expression. Recent studies suggest that phenotypic distributions in isogenic populations could have strong fitness effects, e.g., as bet hedging strategies for rare but toxic environments. Testing such ideas rigorously would benefit from an experimental capacity to specify desired expression distributions and expose them to precisely modulated environments.

To evaluate iCL control for specifying expression distributions, we targeted halves of a population to two nearby CFP fluorescence levels (10, 20 a.u). The experiment ran for 780 minutes in OL mode (precomputed, global light stimulation) to steer expression to the two targets (FIG. 3b). While mean population fluorescence levels initially approach the targets, there are large residual errors in the mean and high variability. Both mean and individual errors interfere with separating the 144 joint population into clearly defined subpopulations with distinct targets (FIG. 3b, center right panel). After 780 minutes, the controllers switched to iCL mode, adjusting stimulation sequences based on individual cell responses (FIG. 3b, bottom). Shortly thereafter, mean error and variation within each subpopulation are sharply reduced. Comparing average mean-subtracted power spectra of individual cells' expression during OL and iCL control, we observe that iCL control reduces amplitudes of slow expression fluctuations (f<1 hr-1; FIG. 3c). The population resolves into two accurately targeted groups of cells (FIG. 3b, rightmost panel). Independent and parallel control of expression in individual cells enables specifying not only stationary distributions, but also precise temporal gene expression patterns in groups of cells. As an example, we used our setup to straightforwardly program a population of 24 cells under iCL control to track distinct dynamic CFP expression targets over a 24 h interval (FIG. 3d).

### Example 3—Controlling Population Variability Through Environmental Antibiotic Perturbation

A key use of our platform is to probe how populations with distributed phenotypes interact with changing environments. Such investigations depend on our ability to modify the environment precisely while maintaining a desired phenotypic distribution. The microfluidic devices we use for long-term culture of individual bacteria are uniquely suited to exert precise chemical and temporal control over cells' environments by switching between media sources. In our setup, we switch media with 1-10 minutes lag at junctions upstream of the device.

For a simple test of combined environmental manipulation and expression control, we elaborated our control experiments to include a switch from antibiotic-free media to media containing a sub-inhibitory level of the translation-inhibiting tetracycline antibiotic, doxycycline (Walsh, Antibiotics: actions, origins, resistance. ASM Press, (2003)). Sub-inhibitory antibiotic concentrations can generate diverse behaviours by modifying gene expression and cell physiology (Aminov, “The role of antibiotics and antibiotic resistance in nature”, Environ. Microbol. Vol. 11, pp. 2970-2988, (2009)). Such changes are difficult to capture in simplified models, and as cells are driven further from the regime in which the control model was identified, the increasing mismatch between model and cell should amplify the mean error between cell fluorescence and control target. Additionally, antibiotics acting on cells with small initial differences in susceptibility could increase variability (and thus mismatch with the average cell) within a population.

We assessed the effect of doxycycline on population and individual control errors by measuring the change in mean and variance of an OL-controlled population with a single fluorescence target (15 a.u.) upon antibiotic exposure. To test how closed-loop control moderates these effects, we assigned a similar number of cells to the iCL control algorithm. We allowed cells 10 hours to acclimatize in the absence of doxycycline before switching to media containing 0.6 ug/ml of the antibiotic, which rapidly slowed the average growth of both OL and iCL-controlled populations by ˜30% (FIG. 4a). Following the growth rate shift, the mean fluorescence of the OL-controlled cells diverges above the target level without stabilizing through an additional 1200 minutes of antibiotic exposure (FIG. 4b). Importantly, because our platform processes cell fluorescence data online, it can detect and respond to effects of changing environmental conditions in real time by appropriately adjusting light inputs. Our simple predictive model of gene expression captured the effects of doxycycline perturbation as an increase in cells' responsiveness, informing the iCL control algorithm that less activating g 183 reen light is required to maintain stable fluorescence levels. The mean fluorescence of the iCL-controlled population thus experiences only a slight, stable increase, without an appreciable increase in population variability (FIG. 4b). The small remaining bias in the iCL-controlled mean results from model mismatch under antibiotic-containing conditions relative to the conditions used for model identification (see SI). Comparison of the probability distributions of OL and iCL-controlled cells in 300 minute intervals prior to and during antibiotic exposure (FIG. 4c) indicates that iCL control largely mitigates both population and individual error, with iCL-controlled expression only slightly affected by the antibiotic.

### Example 4—Hybrid Bio-Digital Circuits in Single Cells

The inventors had the novel insight that besides explicitly controlling gene expression, our platform could digitally virtualize elements of transcriptional circuits. Such bio-digital circuits would permit powerful, facile specification of properties of their digital component (e.g. dynamics, connectivity, response, noisiness), while retaining their in vivo context for assay.

We therefore produced a toy hybrid version of an established circuit, an oscillator, composed of interfaced biological and digital components. Extensively studied natural genetic oscillators exhibit quantifiable phenotypes of frequency, phase, and amplitude (Novak & Tyson, “Design principles of biochemical oscillators” Nat. Rev. Mol. Cell Biol, vol. 9, pp. 981-991, (2008)). Simple synthetic oscillators have been tested as exploratory tools and sensor readouts. A common core architecture among biological oscillators is a delayed negative feedback loop (FIG. 5a), in which a signal (S) produced by a gene product (ENZ) eventually represses the expression of the gene itself. In particular regimes, the signal is then cyclically depleted until expression resumes, producing stable oscillations (Novak & Tyson, “Design principles of biochemical oscillators” Nat. Rev. Mol. Cell Biol, vol. 9, pp. 981-991, (2008)).

We virtualized the signal component, digitally specifying a gene product-dependent production rate (r), first order removal rate (d), and its interaction with the gene's promoter state, P (FIG. 5b). To measure the gene product without producing signal, we replaced the gene with a CFP reporter, thus interfacing the biological to the digital side of the network. CFP fluorescence then governs accumulation of the, now virtual, signal S for each cell. In our toy system, the promoter state is “off” while signal levels exceed a threshold, θ. Last, by replacing the gene's promoter with a light-regulated one that receives virtual promoter states as red/green light stimuli, we interfaced the digital side back to the biological side of the system. We tested our hybrid circuits in single cells, and observed oscillating CFP expression over 40 hours (FIG. 5c, left panel). While the circuits shared similar frequency spectra (FIG. 5c, right panel), variations in individual oscillation lengths shift phases within and between cells. Deterministic behavior of the digital component and low measurement noise suggest that this variability stems largely from the biological side of the circuit.

### Example 5—Freely-Specifiable Digital Communication Between Individual Bacteria

We asked whether re-specifying the digital component could influence the observed variability. Biological oscillators can synchronize by coupling to extracellular fields, which can either be externally imposed or be a product of the local community. For instance, populations of synthetic bacterial oscillators can synchronize through molecular signals that diffuse between cells, forming weakly-coupled transcriptional networks of oscillators (FIG. 5d, top). With this biological architecture in mind, we updated our digital component to define a network of connections between the individual bacteria through which the virtualized signal is redistributed (FIG. 5d, bottom). We repeated the experiment while enforcing communication within cyclically-connected groups of cells by sharing 20% of each cells' signal, Si, between its nearest neighbors. Improved oscillation peak coincidence and nearly zero period-normalized cross-correlation lags indicate that oscillating cells synchronize (FIG. 5e). Given the success of this approach, we explored the behavior of our cells subject to other changes in communication. In our toy system, a freely-specifiable transfer matrix T (FIG. 5d) defines the connection map, and the strengths and signs of interaction between individual cells.

By changing the signs of off-diagonal transfer matrix values, we generated groups of oscillating cells that either inhibit both neighbors' accumulation of signal, or stimulate one neighbor while inhibiting the other. While in our setup this change amounts to a minor change in control software, biological or mechanical implementation of similar operations on bacterial oscillators would be considerably more involved. We tested the new circuits in cyclic, four cell groups. Although we expect the group behaviors to also depend on size and connectivity, patterns in oscillation phases emerge again, but this time with nearest neighbors either a half period, or approximately a quarter period out of phase (FIG. 5 f, g).

### Discussion Examples 1-5

We presented a versatile experimental platform that simultaneously interfaces many individual bacteria with software-defined models in a controlled environment. Directly interweaving ‘wet’ and ‘dry’ components in experiments provides a strong impetus and a ‘test and measurement’ environment for probing predictiveness. By actively testing and adjusting models during experiments, the system could assist in rapid model optimization and facilitate online model inference for single cells.

The platform enables quantitative explorations of individual-based traits of cellular/bacterial populations through feedback control or digitally specified constraints on gene expression in single cells. The demonstrations above illustrate several directions which can be extended to diverse applications. For instance, distributed behaviours can prepare isogenic populations with incomplete sensory information for stochastic environmental variation. Our device enables exploration of this phenomenon by specifying shapes of and dynamics within expression distributions for populations in specified environments. In such a scenario, cells can even be provided with abilities to artificially “sense” the environment via input from the cells' software controllers.

In addition, the paradigm of hybrid genetic circuits formed by interfacing biological and digitally-modelled parts allows biological systems to be characterized under constraint by virtual circuit parts. Analogous to unit testing in software development, this capacity could ease challenges in piecewise debugging of genetic circuits, and in situ development and optimization of synthetic circuits within cell factories. In the latter, in contrast to entirely wet or entirely theoretical approaches, real impacts of virtual components such as sensors could be approximated even before biological versions are engineered, thus speeding up development.

Finally, by specifying hybrid circuits that allow information to flow between individual cells, the system enables new kinds of experiments on how collective behaviours of small groups of bacteria respond to community and environment-dependent changes in cell interactions. Assays of behaviours that respond to digitally defined cell interactions and perturbations (e.g., virtual diffusion or cell arrangement) could assist in exploring interaction-targeted programs for stabilizing, disrupting, or otherwise controlling natural and artificial cellular/bacterial communities.

Individual and group behaviours of microbes are hard to approach fully through simulation alone or simply through biological assay alone. By virtualizing parts of biological systems, our platform achieves benefits of both methods by allowing straightforward specifiability of a virtualized part within its full biological context. Importantly, transplanting digitally-specified components into biological systems can extend the explorable space of circuits and behaviours to and even beyond what is biologically possible.

On the one hand, for characterizing actual genetic circuits within and between cells, the platform can guide (although not replace) traditional fully-biological analyses. On the other hand, its reduced constraints allow us to ask “What if?” questions and to create, perturb, and study approximations to otherwise inaccessible biological systems. Following on the proofs of concept above, our platform can thus facilitate new modes of exploring behaviours of populations and other complex cellular/bacterial systems.

### Methods—Examples 1-5

### Bacterial Strains and Plasmids

The bacterial strains and plasmids used for Examples 1-4 are shown in Table 2. Bacterial strains and plasmids

TABLE 2
Bacterial strains and plasmids

| Name | Genotype | Source |
|---|---|---|
| pSR43.6 | p15A ori, spR, ccaS, ho1-pcyA | Ref. 28 |
| pSR58.6 | colE1 ori, cmR, ccaR, PcpcG2-172sfGFP | Ref. 28 |
| pSR58.6_cerulean | pSR58.6 ΔsfGFP::ceruleanCFP | This work |
| TB201 MG1655 | Pr_venusYFP This work | |
| CR138 | TB201/pSR43.6, pSR58.6_cerulean | This work |
| JW1908 | BW25113 ΔfliC769::kan | KEIO collection⁴⁶ |
| CR141 | TB201 Δ769fliC::frt | This work |
| CR145 | CR141/pSR43.6, pSR58.6_cerulean | This work |

### Imaging and Projection System

Our system integrates image data acquisition and processing, cell stimulation, and environmental regulation. All physical device control and data processing is executed through custom software in MATLAB (version 2014b, with statistics, image processing, distributed computing toolboxes) using MicroManager (Edelsten et al. “Advanced methods of microscope control using uManager software” J. Biol. Methods, vol, 1, 10, (2014)), SUNDIALS (Hindmarsh et al. “SUNDIALS: Suite of nonlinear and differential/algebraic equation solvers” ACM Trans. Math. Softw. TOMS, vol. 31, pp. 363-396, (2005)), mexOpenCV (Yamaguchi. mexopenCV. Available at: https://github.com/kyamagu/mexopencv), and java packages.

Cell growth and expression data is derived from images collected with a motorized inverted microscope (Body: Olympus IX83, Stage:Märzhäuser, Objective:Olympus UPLSAPO100XOPH, Camera: Hamamatsu Orca Flash4.0v2) in the CFP (x438/29,m483/22) and YFP (x513/22,m543/22) fluorescence channels. Software-based focus (modified micro-manager oughtafocus function) is determined at each location/time-point using reflective imaging (475/34 nm) of PDMS-glass interfaces, and a focused reflected image is used for a phase-correlation-based estimate of vertical and horizontal corrections to stage jitter. Fluorescent images are acquired, shading corrected, and cell size and fluorescence-based expression estimates are extracted for individual cells at pre-specified locations within the image. This per-cell data is passed to experiment-dependent software controllers that update cell state estimates and determine the subsequent activation (˜535 nm) or deactivation (˜670 nm) light stimuli to be delivered to each cell.

Light stimuli are simultaneously delivered to cells in a field of view using a variant of a custom modified LCD projector (Stirman et al. “A multispectral optical illumination system with precise spatiotemporal control for the manipulation of optogenetic reagents” Nat. Protoc, vol. 7, pp. 207-220, (2012)). The projector (Panasonic PT-AE6000E) iris is disabled and lamp replaced by 530 nm and 660 nm LED sources (Thorlabs, M530L3,M660L3,LEDD1B). The outer, projection lens is removed and the image projected through the zoom lens is coupled via a field lens (Thorlabs AC508-100-A-ML, f=100 mm) into the rear port of the microscope, through a tube lens (Olympus U-TLU), and via a 50/50 beamsplitter (Thorlabs BSW10R) through the objective and onto the field of view. Projector position is adjusted to bring the camera and projector focal planes into alignment, and sub-micron corrections between the focal planes to be used during the experiment are determined, per channel, at its outset.

The list of per-cell stimuli is converted to red and green boxes in an RGB image, overlying the positions of their corresponding cells. The image is then spatially transformed to register projector to camera image planes (openCV function cv.warpPerspective, using homographies determined at the experiment outset with projected chessboards and the functions cv.findChessboardCorners and cv.findHomography). Shading corrections (low-pass frequency filtered images of reflected flat field projections, generated at experiment setup) are applied to each color channel. The image is then projected onto the field of view and cells illuminated for 10 sec with approximately 10.5 mW/cm2 Red or 7.6 mW/cm2 Green light (with contrast ratios relative to dark LCD panels, of 252 and 361, respectively; crosstalk between channels is less than 1%).

Experimental temperature is regulated within a custom-built opaque, temperature-controlled microscope enclosure via recirculating air heater (controller: CAL3200). Media flow rate is regulated by a pair of syringe pumps (WPI, Alladin-1000).

### Microfluidic Devices

Microfluidic mother machines (23 μm×1.3 μm×1.3 μm (l,w,h) growth channels with 5 μm spacing along split media trench) are fabricated by curing degassed polydimethylsiloxane (Sylgard 184, 1:10 catalyst:resin) against epoxy replicate master molds produced from primary wafer-molded devices (Estevez-Torres et al, “An inexpensive and durable epoxy mould for PDMS” Chips and Tips. (2009) and Bergmiller et al. “Biased partitioning of the multidrug efflux pump AcrAB-TolC underlies long-lived phenotypic heterogeneity” Science. Vol, 356, pp. 311-315, (2017)). Cured PDMS bulk is peeled from the molds and trimmed as appropriate, input and output ports are punched with electropolished 18 ga luer stubs. The PDMS bulk and a clean cover slip are rinsed with 100% isopropanol, blown dry, baked on a hotplate at approximately 125 C for 15 minutes. They are then cooled, and the surfaces to be bonded exposed to air plasma (326 Harrick PDC-002 plasma cleaner, medium power) for 1 minute, and then brought gently into contact. The bonded devices are left at room temperature for 15 minutes, post-baked for 1-2 hours at 80 C, and then stored until use. Polyethylene tubing (Instech, BTPE-50) is press-fitted onto 22 ga luer stubs and cannulae (Instech) on opposite ends for coupling to media supplies and waste, and to the devices, respectively.

### Experiment Setup and Conditions

A frozen glycerol cell stock is thawed from −80 C, diluted 1:100 into 5 ml fresh LB containing 0.01% Tween20, with 20 μg/ml Chloramphenicol and 100 μg/ml Spectinomycin to maintain plasmids, and incubated for 6-7 hours at 37 C. The experimental apparatus is initialized, prewarmed and equilibrated, and the microfluidic device flushed for 1 minute with 0.01% Tween20 followed by air. The device is mounted to the microscope stage to warm and verify integrity. The grown cell culture is centrifuged at 4000×g for 4 minutes, and the pellet resuspended in a few μ1 supernatant and injected into the device by pipette. Once filling of the growth channels with cells is confirmed under the microscope, media supply and waste tubes are fitted to the device and running media (LB, 0.4% glucose, 0.01% Tween20) is flowed through the device at 4 ml/hour for 1 hour, and 1.5 ml/hour-2.0 ml/hour thereafter. The experiment control software is engaged. Experiment calibration, providing per-channel camera and projector offsets from the PDMS-glass interface focal plane, projector-camera image transforms, and projector shading correction are performed. For each control location on the chip, measurement areas for individual cells are specified (typically, by a 2.6×5.2 μm box at the end of a growth channel), and a software controller/target program is associated with each. Once all control locations have been populated and the system begins to acquire data and stimulate the cells, it runs automatically until the conclusion of the experiment. For experiments involving media switching, a stopcock and syringe pump flows are adjusted at the appropriate time (0.3 ml media plug between the stopcock and cells is replaced after approximately two 6-minute cycles).

### Media

LB containing 0.01% Tween20, 100 μg/ml Spectinomycin, and 20 μg/ml Chloramphenicol is used for strain preculture and plasmid maintenance preceding insertion of cells into the device. Running media (LB, 0.01% Tween20, 0.4% Glucose) is used thereafter. For doxycycline perturbations, a 1 μg/ml stock solution of doxycycline is diluted in running media to a final experimental concentration, and maintained at 23 C in the dark from the start of the experiment until use.

### Cell Validation

At some rates, bacterial cells in mother machine devices can filament, shift spatially and even escape growth channels, or stop growing. Optogenetic systems are also subject to mutational dysfunction and plasmid loss from cells. To reduce noise injection and remove pathological cells and locations from our experiments, the mother cells in our device are automatically evaluated for continuous presence, growth, and maintenance of the optogenetic system. We use the constitutively-expressed YFP to verify cell presence (any YFP signal loss immediately invalidates growth channels), and to extract cell outlines and invalidate non-growing cells whose smoothed elongation rate has fallen below a minimum threshold. Besides growth arrest, loss of the colE1-based plasmids can occur, corresponding with a substantially increased elongation rate and reduced YFP level, which invalidate cells at threshold crossings. For analysis, data from invalidated cells is truncated 150 minutes before threshold violations.

Infrequent, complete loss of green light response is typified by unrecoverable dilution of CFP signal to zero. To avoid invalidating transiently unresponsive cells, otherwise valid cells that sustain this phenotype without recovery are manually removed during experiment analysis.

### Example 6

### Introduction

This example describes how to rapidly implement a network of synthetic oscillators in Chinese Hamster Ovary (CHO) cells capable of synchronizing to each other by a recently proposed quorum sensing mechanism. The model is based on the synthetic mammalian oscillator of Tigges et al. “A tunable synthetic mammalian oscillator” Nature. Vol, 457, pp. 309-312, (2009), which describes in detail the proposed genetical implementation and the respective ordinary differential equations of the model.

The core oscillator (see FIG. 6a and Tigges et al.) consists of two proteins, the tetracycline-dependent transactivator (tTA) and the pristinamycin-dependent transactivator (PIT), and their respective mRNAs and a tTA antisense mRNA. Both the transcription of tTA mRNA and PIT mRNA is driven by the tTA protein. The transcription of the tTA antisense mRNA is in turn driven by the PIT protein. The tTA antisense mRNA can bind to the tTA sense mRNA and thus deactivate its translation. The ability for intercellular communication, and thus for synchronization, is achieved by an additional feedback loop utilizing the quorum sensing mechanism of the marine bacterium Vibrio fisher (Schaefer et al. “Generation of cell-to-cell signals in quorum sensing: Acyl homoserine lactone synthase activity of a purified Vibrio fischeri luxi protein” Proc. Natl. Acad. Sci, vol. 93, pp. 9505-9509, (1996)), consisting of two genes encoding the sender protein LuxI, and the receptor protein LuxR.

The transcription rates of the LuxI and the LuxR genes depend on the phase of the core oscillator through the PIT transcriptional activator. Since the LuxI protein synthesizes the autoinducer (3OC6HSL, a small signaling molecule), its concentration will oscillate with the same frequency as the core oscillator, but with a phase shift depending on the dynamics of the transcription, translation and degradation of LuxI and on the production and degradation rate of the autoinducer. The autoinducer can freely diffuse through the cell membrane and cells can thus obtain information about the phase of other cells surrounding them. LuxR and the autoinducer form a complex that dimerizes and can be used as a transcriptional activator for additional genes. The gene of the antisense mRNA was combined with a promoter activated by the dimerized receptor-autoinducer complex.

To rapidly engineer this oscillatory network, the whole network could be divided into two modules, an oscillatory and a communication module (FIG. 6b). The oscillatory module consists of the core oscillator as implemented in Tigges et al., consisting of the tTA, PIT and antisense genes. The input for this module is constructed by putting an additional antisense gene under the control of the GAL4 UAS-promoter. The output for this model is constructed by fusing a fast maturating fluorescence protein to PIT thus being able to detect its concentration in real time.

The second module which realizes the communication mechanism consists of the genes for LuxI and LuxR. To be able to change the input of this module the promoters of both genes are exchanged by the GAL4 UAS-promoter. As an output for this second module, an additional gene may be added encoding a fast maturating fluorescence protein, which is under the control of the promoter being activated by the LuxR-antisense dimer. Furthermore, in the cells realizing both modules, the genes phyB-GBD and PIF3-GAD (Mendelsohn. “An englightened genetic switch” Nature Biotechnology. Vol. 20, pp. 985-987, (2002)) may be inserted, and used for the light-inducible transcription mechanism.

Since both modules are implemented in separate cells, the same fluorescence marker may be used as the output signal and the same light inducible transcription unit as input. This separation into two modules enables analysis of the oscillatory and the communication module separately. By putting the cells for the communication module in a microfluidic device the concentration of the extracellular autoinducer can additionally be controlled, thus simulating different cell densities and synchronization stages of a population of synchronizing cells.

The separation into the described modules is interesting for faster validation and error correction of the single modules. The different properties that determine if a population of chemically coupled cells will synchronize are distributed between the two modules: The oscillatory module can be used to test and increase the sensitivity of the phase of the oscillator to oscillatory inputs, whereas the communication module can be used to determine the effect of cell density and to adjust signal strength to guarantee good synchronization results. The third property that determines if a population of chemically coupled cells will synchronize—cell diversity—affects both modules. The strength of cell diversity can be determined by repeating the same experiment multiple times simultaneously in different wells of a microplate.

### Discussion

We propose a method to rapidly test, model or engineer biological circuits by taking advantage of the modular design inherent in most synthetic networks. Single modules are implemented separately in vivo and their proper functionalities in the context of the overall network are tested by simulating the rest of the network in silico. The output signals of a module are read out in real time during the experiment, in the present example by using fluorescence proteins and quantifying the value of fluorescence using cell detection and cell tracing combine with parameter and state estimation. The output of the module is used for the simulation of the rest of the network. The outcome of the simulation is fed back to the in vivo module, in the present example using light-switchable promoters or light-inducible degradation. After validating the proper dynamic behaviour of every module in this way, the single modules may be combined into the overall network. This method decreases thus the amount of iteration cycles of the rational design cycle, thus decreasing the cost and time for de novo implementations of synthetic biological networks.

### Example 7

This example describes work to reconstruct a reliable implementation of the “Repressilator” (Elowitz & Leibler (2000), Nature, 403(6767), 335). While this comparatively simple oscillatory network formed the foundation of synthetic biology almost 20 years ago, its reliable implementation is still a matter of current research. Specifically, the Repressilator corresponds to a negative feedback loop composed of three genes each encoding a repressor. Each individual repressor thereby represses the expression of the subsequent gene. The “intended functionality” of this network is to show regular oscillations over many cell generations.

In order to apply the methodology described herein to this network, the Repressilator was decomposed into three units, each consisting of the gene encoding for one of the repressors as well as the respective downstream promoter. Based on these three units, a total 13 different bio-digital constructs were created: (i) three constructs consisting of each biological unit in isolation combined with our light system to test these units; (ii) three constructs representing “meta-units” each composed of two units interfaced with the light system; (iii) three constructs consisting of all three units, where the feedback is “interrupted” between two repressors and replaced by our light system (thus corresponding to three step repressor cascades); (iv) three complete Repressilators with expression reporters for each repressor; and (v) a diagnostic light system reporter with a complete Repressilator in the background. Together, these constructs correspond to all possible ways the complete Repressilator (and light system) can be (fully or partially) assembled from its underlying units. The intention is to exhaustively implement the Repressilator for demonstration and testing purposes. However, for other purposes it is anticipated that it will only be necessary to construct a subset of all possible units.

Data from testing exemplary individual units that have been successfully implemented and validated in isolation are shown in FIG. 7. The in vivo implementation of each individual unit in real time can then be connected with an in silico simulation of the rest of the network to test if each individual unit is compatible with the functionality of the overall network, given that the rest of the network operates according to specification. Next integration tests will determine whether two or more units together still lead to the intended functionality when combined. Successive integrations of biological components can continue until the entire network is successfully implemented. In each step, for any inconsistencies of the dynamic properties of each individual unit or combinations of units identified by the bio-digital framework, investigations can be made to localize the underlying biological reasons for the inconsistency, and home in on improved implementations.

1. A system for engineering, testing or modelling a biological circuit, the system comprising
(i) means for culturing one or more living cells;
(ii) one or more living cells in which a cellular part of the biological circuit is implemented;
(iii) means for controlling the state or environment of the cell(s);
(iv) means for taking measurements of one or more cell state or environmental parameters from the cell(s); and
(v) a computer;
characterised in that the computers simulates the remaining part of the biological circuit in real time; and the two parts interface via a closed loop in which the output from the simulation provides input into the cellular part of the biological circuit via the controlling means; and cell state or environmental parameter measurements taken from the cell(s) provide input into the simulated part of the circuit.

2. The system of claim 1, comprising a population of living cells in which a part of the biological circuit is implemented, wherein the means for controlling the state or environment of the cells are set based on measurements of one or more cell state or environmental parameters taken from one or more other cells in the population.

3. The system of claim 2, wherein the system comprises means for differentially controlling the state or environment of different cells in the population and intercellular communication between cells of the population is virtualised by feeding-back to individual cells or groups of cells, via the means for differentially controlling the state or environment of different cells in the population, with input determined by measurements of one or more cell state or environmental parameters taken from one or more other individual cells or groups of cells of the population.

4. The system of claim 2, further comprising one or more living or virtual mutant cells for perturbing the biological circuit, wherein a part of the biological circuit operates in the mutant cell(s) and is different from that implemented in the other, non-mutant cells.

5. Use of the system of claim 1 for engineering, testing or modelling a biological circuit.

6. A method of engineering, testing or modelling a biological circuit, the method comprising
(i) culturing one of more living cells, wherein a cellular part of the biological circuit is implemented in the cell(s);
(ii) taking measurements of one or more cell state or environmental parameters from the cell(s);
(iii) simulating the remaining part of the biological circuit in real time on a computer; and
(iv) interfacing the cellular and simulated parts of the circuit via a closed loop in which the output from the simulation provides input into the cellular part of the biological circuit via means for controlling the environment or state of the cell(s); and cell state or environmental parameter measurements taken from the cell(s) provide input into the simulated part of the biological circuit.

7. The method of claim 6, comprising culturing a population of living cells in which a part of the biological circuit is implemented; and feeding back to cells in the population, via the means for controlling the environment or state of the cells, with input that is determined by measurements of one or more cell state or environmental parameters taken from one or more other cells in the population.

8. The method of claim 7, further comprising virtualising intercellular communication between cells of the population by feeding-back to individual cells or groups of cells, via means for differentially controlling the environment or state of different cells in the population, with input that is determined by measurements of one or more cell state or environmental parameters taken from one or more other individual cells or groups of cells of the population.

9. The method of claim 7, further comprising introducing into the cell population one or more living or virtual mutant cells for perturbing the biological circuit, wherein a part of the biological circuit operates in the mutant cell(s) and is different from that implemented in the other, non-mutant cells.

10. The method of claim 6, comprising engineering, testing or modelling a first biological circuit according to the method of claim 6, and further engineering, testing or modelling a second biological circuit according to the method of claim 6, wherein the second biological circuit is a modified version of the first biological circuit.

11. The method of claim 6, comprising engineering, testing or modelling a first biological circuit according to the method of claim 6, modifying the cellular part and/or the simulated part of the biological circuit, and repeating the method of claim 6 to engineer, test or model the modified biological circuit.

12. The method of claim 10, wherein the modification comprises implementing in the cellular part of the second biological circuit an element that was simulated by the computer in the first biological circuit.

13. The method of claim 10, wherein the modification comprises simulating in the computer an element of the second biological circuit that was implemented in the living cell(s) in the first biological circuit.
